(19) United States
(12) Patent Application Publication
(10) Pub. No.: US 2022/0096077 A1
(43) Pub. Date: Mar. 31, 2022

(54) SUTURE PASSING DEVICE

(71) Applicant: ARCH DAY DESIGN, LLC, Ventura, CA (US)

(72) Inventors: Roger PISARNWONGS, Santa Clarita, CA (US); Thomas WEISEL, Ventura, CA (US)

(21) Appl. No.: 17/447,396

(22) Filed: Sep. 10, 2021

Related U.S. Application Data
(60) Provisional application No. 63/198,085, filed on Sep. 28, 2020.

(51) Int. Cl. A61B 17/04

(52) U.S. Cl. CPC A61B 17/0482; A61B 2017/00367; A61B 17/0469

(57) ABSTRACT

A suture passing device includes a jaw assembly having both a loose suture capturing mechanism and a tight suture grasping mechanism. The jaw assembly includes a pair of jaw members biased to a default diverging configuration. At least one jaw member includes a hook that defines a closed loop and a dead-end channel for loosely receiving suture when the jaw assembly is in the closed position. The jaw assembly is formed at a distal portion of a thin bendable ribbon.

### CROSS-REFERENCE TO RELATED APPLICATION(S)

This application claims the benefit of priority of U.S. provisional patent application No. 63/198,085, filed Sep. 28, 2020, the contents of which are herein incorporated by reference.

### FIELD OF THE INVENTION

The present invention relates in general to medical devices. More particularly, the invention is directed to a suture passer.

### BACKGROUND OF THE INVENTION

Often times an important aspect of a surgical procedure is to pass suture through tissue. This could be required to mend a tear or connect two or more pieces of soft tissue. Though this task is common it can be challenging for the surgeon especially in an arthroscopic procedure where visualization is limited.

There are still certain procedures and anatomies where the average surgeon still struggles. For example, suturing the labrum at times can prove challenging particularly when the tissue is severely damaged. In such cases, a large overall profile of the feature penetrating the tissue would make suturing particularly challenging.

A needle with a smaller profile would also make it challenging to house a mechanism that can pass or retrieve suture in an arthroscopic atmosphere. With many devices currently on the market, a very small loop of suture is provided during tissue passing such that the surgeon is challenged to hit the miniscule target and retrieve the suture.

### SUMMARY OF THE INVENTION

In one aspect, a suture passing device comprises a handle, a shaft coupled to the handle, a jaw assembly housed within shaft, a suture grasping mechanism and a hook formed on jaw member. The shaft comprises a sharp distal tip that defines a shaft axis, while the shaft defines a lumen. The jaw assembly comprises a pair of jaw members including a first jaw member and a second jaw member. The first jaw member and the second jaw member are movable with respect to each other between a closed position and an open position.

An actuating mechanism is coupled to the jaw assembly and configured to move the jaw assembly between the closed position and the open position. The first jaw member and the second jaw member are biased away from each towards the open position and moved to the open position when the actuating mechanism moves the jaw assembly distally with respect to the shaft such that first jaw member and the second jaw member each diverge from the shaft axis when exiting the shaft. The first jaw member and the second jaw member are moved to the closed position when the actuating mechanism is moved to retract the jaw assembly proximally with respect to the shaft.

The hook defines a channel for receiving a portion of a suture. The channel is sized to loosely carry suture. The hook may form a closed loop with a distal tip of the second jaw member when the jaw assembly is in the closed position.

The first jaw member may preferably comprise a first width in the range of 0.2 mm to 1 mm. The second jaw member may preferably comprise a second width in the range of 0.2 mm to 1 mm.

The second leg may comprise a leg extension with a leg extension width smaller than the second width.

In a further embodiment, the second jaw member may comprise a second hook.

The first jaw member may comprise a first proximal portion having a first scalloped edge. The second jaw member may comprise a second proximal portion having a second scalloped edge or a flat edge.

If the jaws do not have scalloped edges, the suture grasping mechanism may comprise a first proximal jaw portion having a first flat surface, a second proximal jaw portion having a second flat surface that contacts the first flat surface when the jaw assembly is in the closed portion, and a crimped shaft portion proximal to the sharp distal tip.

In another aspect, a suture passing device is provided with a hook and a suture grasping mechanism. The device comprises a handle, a shaft coupled to the handle, and a jaw assembly. The shaft comprises a sharp distal tip that defines a shaft axis, the shaft defining a lumen. Housed within the shaft, the jaw assembly comprises a pair of jaw members including a first jaw member and a second jaw member. The first jaw member and the second jaw member are movable with respect to each other between a closed position and an open position. An actuating mechanism is coupled to the jaw assembly and configured to move the jaw assembly between the closed position and the open position

The first jaw member and the second jaw member are biased away from each towards the open position and moved to the open position when the actuating mechanism moves the jaw assembly distally with respect to the shaft such that first jaw member and the second jaw member each diverge from the shaft axis when exiting the shaft. The first jaw member and the second jaw member are moved to the closed position when the actuating mechanism is moved to retract the jaw assembly proximally with respect to the shaft. The first jaw member comprises a hook that forms a closed loop with a distal tip of the second jaw member when the jaw assembly is in the closed position.

The suture grasping mechanism may comprise features formed on the jaw members, such as scalloped edges. Alternatively, the suture grasping mechanism may comprise a combination of the jaw assembly working in conjunction with a crimped shaft portion to securely grasp suture. For example, the suture capturing mechanism may comprise a first proximal jaw portion having a first flat surface, a second proximal jaw portion having a second flat surface that contacts the first flat surface when the jaw assembly is in the closed portion, and a crimped shaft portion proximal to the sharp distal tip.

The first jaw member may preferably comprise a first width in the range of 0.2 mm to 1 mm. The second jaw member may preferably comprise a second width in the range of 0.2 mm to 1 mm.

The second jaw member may comprise a second hook that is laterally offset with respect to the first hook.

The first jaw member may comprise a first proximal portion having a first scalloped edge. The second jaw member may comprise a second proximal portion having a second scalloped edge.

In a further aspect, a suture passing device is provided with wider jaw members. The device comprises a handle and a shaft coupled to the handle. The shaft defines a lumen and comprises a sharp distal tip that defines a shaft axis. A jaw assembly is housed within the shaft and comprises a pair of jaw members including a first jaw member and a second jaw member. The first jaw member and the second jaw member are movable with respect to each other between a closed position and an open position.

The first jaw member and the second jaw member are biased away from each towards the open position and moved to the open position when the actuating mechanism moves the jaw assembly distally with respect to the shaft such that first jaw member and the second jaw member each diverge from the shaft axis when exiting the shaft. The first jaw member and the second jaw member are moved to the closed position when the actuating mechanism is moved to retract the jaw assembly proximally with respect to the shaft.

An actuating mechanism is coupled to the jaw assembly and configured to move the jaw assembly between the closed position and the open position.

The device comprises a suture sliding mechanism and a suture grasping mechanism configured to grasp tightly onto suture.

The first jaw member comprises a hook that forms a closed loop with a distal tip of the second jaw member when the jaw assembly is in the closed position. This closed loop, or closed-end channel, comprises the suture sliding mechanism.

The first jaw member may comprise a first width in the range of 0.2 mm to 1 mm. The second jaw member may comprise a second width in the range of 0.2 mm to 1 mm.

The second jaw member may comprise a second hook that is laterally offset with respect to the first hook.

The first jaw member may comprise a first proximal portion having a first scalloped edge. The second jaw member may comprise a second proximal portion having a second scalloped edge.

If the jaw members do not have scalloped edges, the suture grasping mechanism may comprise a first proximal jaw portion having a first flat surface, a second proximal jaw portion having a second flat surface that contacts the first flat surface when the jaw assembly is in the closed portion, and a crimped shaft portion proximal to the sharp distal tip.

Methods of passing suture are also provided.

### BRIEF DESCRIPTION OF THE DRAWINGS

FIG. 1 is a top perspective view of a suture passing device according to first preferred embodiment.

FIG. 2 is a close-up view of a first preferred jaw assembly in an open configuration.

FIG. 3 is close-up view of the first preferred jaw assembly in a closed configuration.

FIG. 4 is a top plan view of a second preferred embodiment of the suture passing device with a second preferred jaw assembly in an open configuration.

FIG. 5 is a side elevation view of the second preferred embodiment of the suture passing device with jaw assembly in the open configuration.

FIG. 6 is an axial cross-sectional view of the second preferred embodiment of the suture passing device.

FIG. 7 is a top plan view of the second preferred embodiment of the suture passing device.

FIG. 8 is an axial cross-sectional view of the second preferred embodiment of the suture passing device.

FIG. 9 is a close-up view of the second preferred jaw assembly in the open configuration.

FIG. 10 is a close-up view of the second preferred jaw assembly in the closed configuration.

FIG. 11 is an operative view of the second preferred embodiment of the suture passing device in a fully retracted configuration.

FIG. 12 is an operative view of the second preferred embodiment of the suture passing device with the jaw assembly in an open configuration.

FIG. 13 is an operative view of the second preferred embodiment of the suture passing device with the jaw assembly in a partially retracted position.

FIG. 14 is a close-up operative view of the second preferred embodiment of the suture passing device with the jaw assembly in the partially retracted position loosely carrying captured suture.

FIG. 15 is an operative view of the second preferred embodiment of the suture passing device with the jaw assembly loosely carrying captured suture through tissue.

FIG. 16 is an operative view of the second preferred embodiment of the suture passing device with the jaw assembly in the open configuration to release captured suture after traversing tissue.

FIG. 17 is an operative view of the second preferred embodiment of the suture passing device with the jaw assembly in the open configuration to engage suture.

FIG. 18 is a close-up axial view of the second preferred embodiment of the suture passing device with the jaw assembly in a retracted closed position with suture captured therein.

FIG. 19 is an operative view of the second preferred embodiment of the suture passing device with captured suture being inserted through tissue.

FIG. 20 is an operative view of the second preferred embodiment of the suture passing device with the jaw assembly deployed to release suture.

FIG. 21 is a perspective view of a third preferred jaw assembly.

FIG. 22 is a further perspective view of the third preferred jaw assembly.

FIG. 23 is an elevation view of the third preferred jaw assembly.

FIG. 24 is a perspective view of a fourth preferred jaw assembly.

FIG. 25 is an elevation view of a fourth preferred jaw assembly.

FIG. 26 is an elevation view of an alternate preferred embodiment with a curved needle shaft.

FIG. 27 is a perspective view of an alternate preferred embodiment of a suture passing device having a suture capturing mechanism external to the jaw assembly.

FIG. 28 is a perspective view of a top plan view of the jaw assembly of the alternate preferred embodiment of a suture passing device of FIG. 27.

FIG. 29 is a diagram of a method of passing suture.

### DETAILED DESCRIPTION OF THE PREFERRED EMBODIMENTS

The following preferred embodiments, in general, are directed to devices and methods for manipulating and passing suture. As will be appreciated, aspects of the suture passing device and its embodiments provide convenience for grasping suture and passing captured suture through tissue. Moreover, aspects disclosed are useful and superior to conventional suture passing devices because the preferred elements provide a reliable and more convenient capture of sutures.

In general, preferred devices include a jaw assembly that captures suture loosely or securely. It will be understood that the embodiments disclosed may include different jaw assemblies and suture capturing mechanisms in combination and no one jaw assembly is necessarily operated with any particular suture capturing mechanism disclosed. Thus enumeration in the following does not imply that a jaw assembly with the same number series must be operated by a suture capturing mechanism of the same number series.

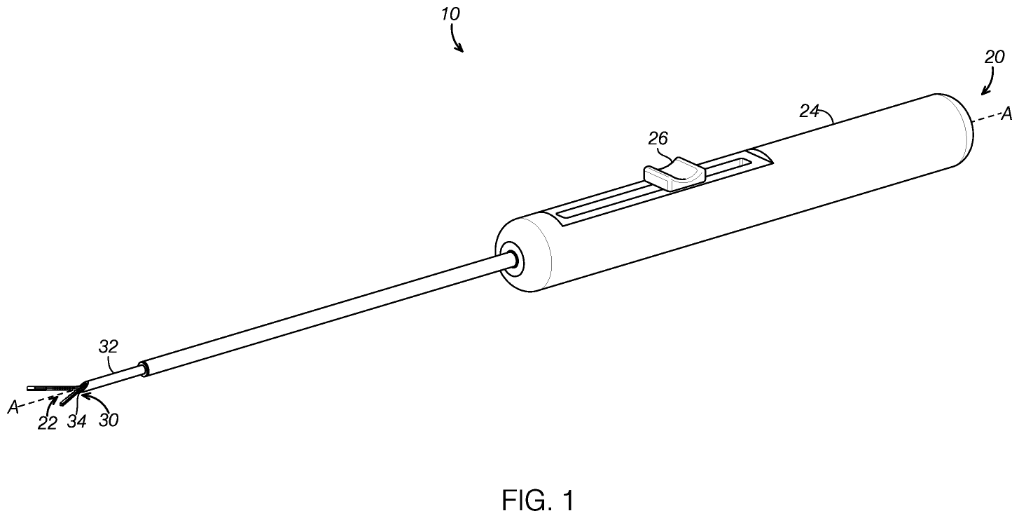

Referring now to FIG. 1, a first preferred embodiment of a suture passing device or suturing device 10 is shown. The device 10 preferably comprises a proximal portion 20 and a distal portion 22. The proximal portion 20 may include a handle 24 and an actuating mechanism 26 such as a thumb slide. The actuating mechanism 26 communicates with and actuates a jaw assembly 30 located near the distal portion 22 of the device 10. An actuator (not shown) is coupled to the thumb slide and the jaw assembly 30 which is housed in a shaft 32. The shaft 32 defines a shaft axis A.

The shaft 32 comprises a distal tip 34. In the preferred embodiment, the distal tip 34 comprises a sharp needle tip 34 that enhances its ability to puncture tissue. While the distal tip 34 is shown in a straight configuration for simplicity, it should be expressly understood that this tip can be bent in a number of different curves as required.

In the preferred embodiment, the jaw assembly 30 is formed at the distal portion of a thin ribbon 38 that can traverse through straight or curved shafts as discussed further below. The ribbon 38 preferably has a thickness in the range of 0.2 mm to 1.0 mm.

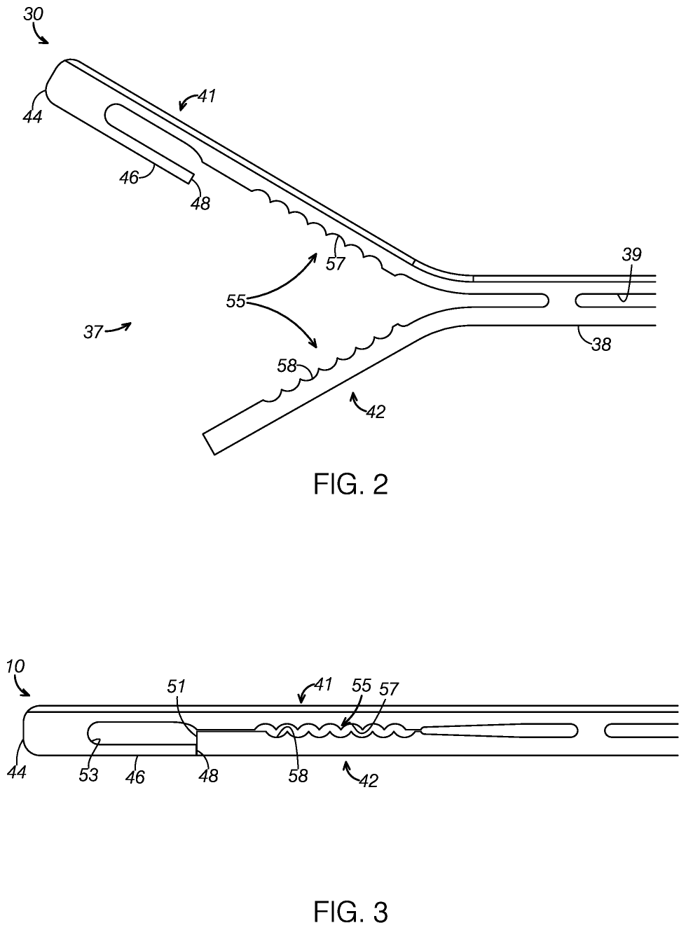

FIG. 2 shows a close-up of the first preferred jaw assembly 30 that is formed at the distal portion 37 of a thin ribbon 38. In the preferred embodiment, the ribbon 38 is substantially flexible and bendable such that it can traverse through straight or curved shafts as discussed further below. The ribbon 38 preferably has a thickness in the range of 0.2 to 1.0 mm. The ribbon may also comprise proximal cutouts 39, or the absence of material, to increase flexibility, enabling the ribbon 38 to bend easily.

It will be appreciated that the suture passing device 10 is configured to capture suture either loosely or securely, i.e., at a fixed point of suture. The suture passing device 10 thus engages suture in two ways:
1) a sliding mode where suture is loosely carried by the device 10 and slide with respect to the jaw assembly 30; or
2) a grasping mode where suture is held securely at a fixed point by the device 10, and the grasped portion of suture moves in tandem with the device 10.

In the preferred embodiment, the suture passing device 10 provides the above dual modes of suture engagement while comprising a substantially thin ribbon that may bend through curved pathways (e.g., curved shaft).

In FIG. 2, the illustrated jaw assembly 30 is shown in a default open configuration which occurs when the actuating mechanism 26 of FIG. 1 is moved distally to cause the jaw assembly 20 to exit the shaft distal tip 34. In the first preferred embodiment, the jaw assembly 30 preferably comprises a pair of jaw members, namely, a first jaw member 41 and a second jaw member 42.

The first jaw member 41 preferably comprises a distal hook 44 configured to loosely retain and carry suture. In the preferred embodiment, the hook 44 comprises a return 46 that extends in a proximal direction and terminates at a proximally facing end 48. When the jaw assembly is in the open position as shown in FIG. 2, the hook 44 may loosely capture or “hook” onto suture in a loose manner without requiring assistance from the second jaw member 42.

When the jaw assembly 30 is in the closed position as shown in FIG. 3, the abutting end 48 of the first jaw member 41 abuts the distal end 51 of the second jaw member 42 to form a closed-end channel, or dead-end channel, 53. The closed-end channel 53 forms a closed loop that prevents suture captured therein from axially (i.e., in a distal or proximal direction) escaping the device. Thus, the closed-end channel 53 therefore comprises a suture sliding mechanism that enables engaged suture to slide with respect to the device 10.

In FIGS. 2 and 3, the jaw assembly 30 preferably comprises a suture grasping mechanism 55 configured to capture suture securely, or tightly, by grasping onto a fixed point of the suture. In the illustrated embodiment, the suture grasping mechanism 55 comprises a first scalloped edge 57 formed on a medial side of the first jaw member 41 and a second scalloped edge 58 formed on an opposite medial side of the second jaw member 24. The pair of opposed scalloped edges 57, 58 are aligned with each other so as to fix on a point of secured suture when the jaw assembly 30 is in the closed position as shown in FIG. 3.

Alternatively, the device 10 may comprise a single scalloped edge formed on jaw member and a flat surface formed on the opposing jaw member.

The suture grasping mechanism 55 works in contrast to the hook 41 which loosely carries suture within its closed-end channel 53. It will be appreciated that the jaw assembly 30 preferably comprises both a loose suture capturing mechanism 41 and a tight suture capturing mechanism 55. In the preferred embodiment, the tight suture grasping mechanism 55 is located proximally to the loose suture carrying mechanism 41. This dual arrangement enables the distal portion of the jaw assembly 30 to carry suture loosely and the proximal portion of the jaw assembly 30 to grab onto suture tightly. This distal-loose and proximal-tight arrangement of the jaw assembly 30 may be preferable for a surgeon in maneuvering the suture passing device 10 to first encounter and engage suture loosely, and then to grasp onto the suture with a more proximal section of the jaw assembly 30.

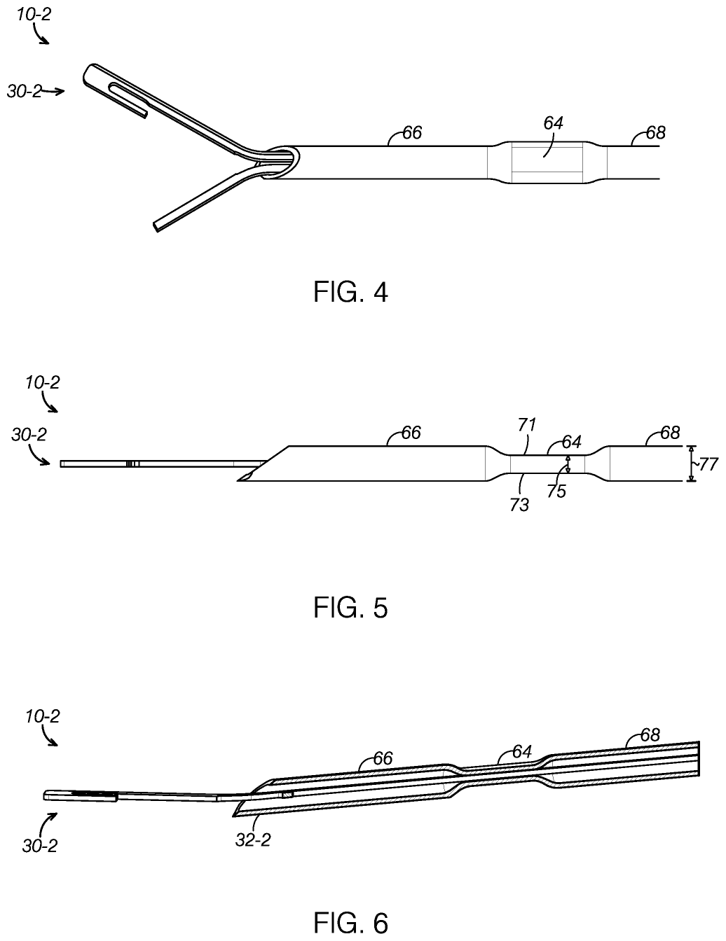

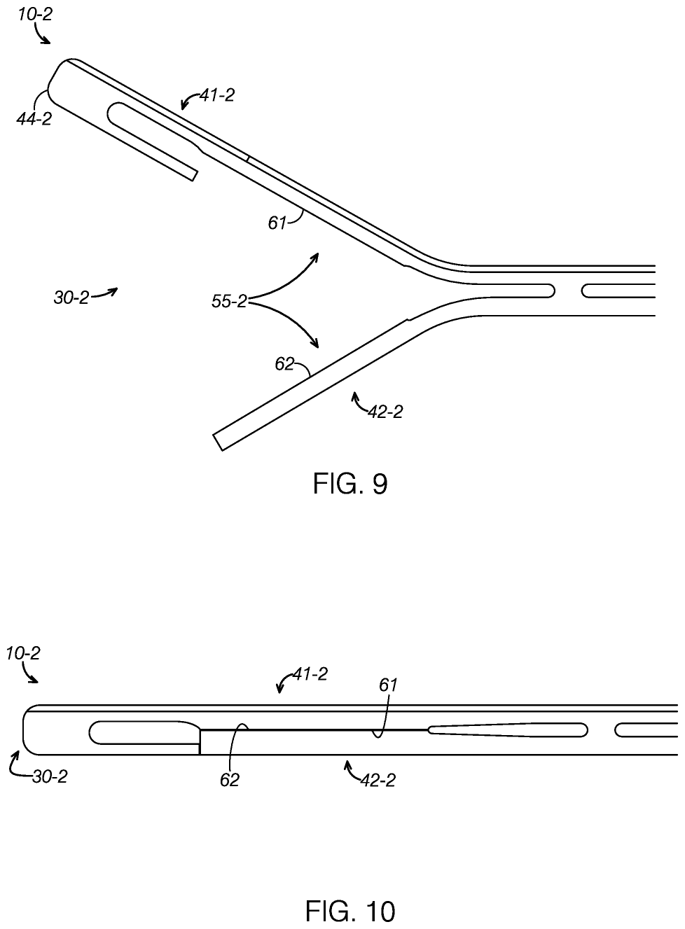

FIGS. 4-6 illustrate a second preferred embodiment of a suture passing device 10-2 having a jaw assembly 30-2 that includes loose and tight suture capturing mechanisms. In FIG. 9., the jaw assembly 30-2 comprises a suture grasping mechanism 55-2 that includes a pair of opposed substantially flat medial surfaces 61, 62 that clamp down on a fixed point of suture when the jaw assembly 30-2 is in the closed position.

The second preferred suture passing device 10-2 includes a shaft 32 with a crimped portion 64 disposed between a distal shaft portion 66 and a proximal shaft portion 68. The crimped portion 64 comprises a first indented surface 71 and an opposite second indented surface 73. As shown in FIG. 5, the indented surfaces 71, 73 define a cross dimension 75 that is less than a diameter 77 of the adjacent shaft portions 66, 68.

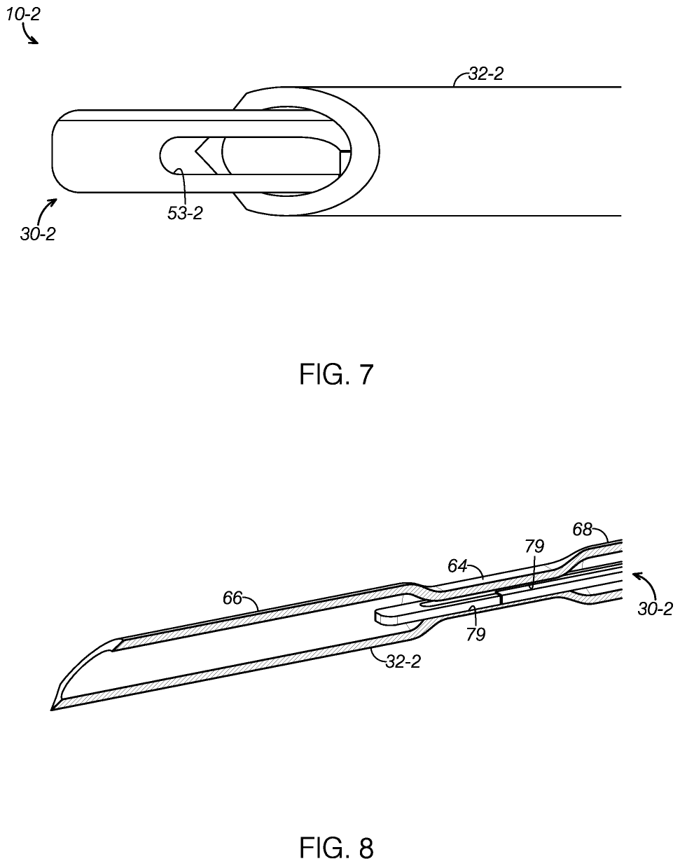

As shown in FIG. 6, the jaw assembly 30-2 is in an open configuration as it extends out from the shaft 32-2. In FIG. 7, As the jaw assembly 30-2 is retracted into the shaft, the jaw assembly 30-2 closes to form the closed-end channel 53-2. In FIG. 8, as the jaw assembly 30-2 is further retracted within the shaft 32-2, the jaw assembly 30-2 slides through the crimped portion 64 to form a snug fit with the inner flat walls 79 of the crimped portion 64.

FIGS. 9-10 show close-up views of the suture grasping mechanism 55-2 of the jaw assembly 30-2. As shown more clearly in FIG. 18, the suture grasping mechanism comprises a pair of opposed flat surfaces 61, 62 and a crimped shaft portion 64 that works in conjunction with the pair of opposed flat surfaces 61, 62. When the jaw assembly is in the closed position as shown in FIG. 5, the first flat surface 61 on the first jaw member 41-2 abuts the second flat surface 62 on the second jaw member 42-2

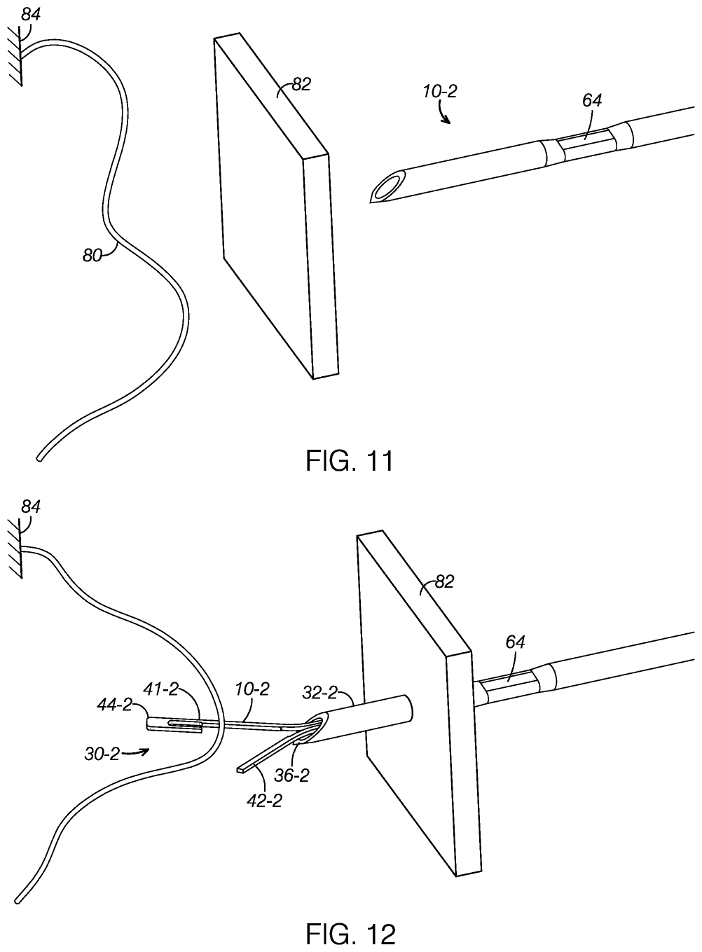

FIGS. 11-16 illustrate a method of retrieving suture using the second preferred embodiment of the suture passing device 10-2, particularly when one end of the suture 80 is fixed to other tissue or implanted hardware at a fixation point 84. In such situations where one end of the suture 80 is fixed at a certain point, it may be preferable to loosely engage the suture 80 and carry the suture 80 through a layer of tissue 82 without grasping the suture. This loose retrieving of a suture attached at one end allows the captured suture to slide with respect to the device 10-2 as the suture is being carried through a layer of tissue. In FIG. 12, the needle 36-2 of the shaft 32-2 has pierced the tissue 82 and the jaw assembly 30-2 has been actuated distally with respect to the shaft 32-2 to exit the shaft 32-2 and move to its default open configuration.

In this open position, the first jaw member 41-2 with the hook 44-2 can be maneuvered to retrieve a portion of the suture 80 while the second jaw member 42-2 is spaced a comfortable distance from the first jaw member 41-2 so as not to get in the way.

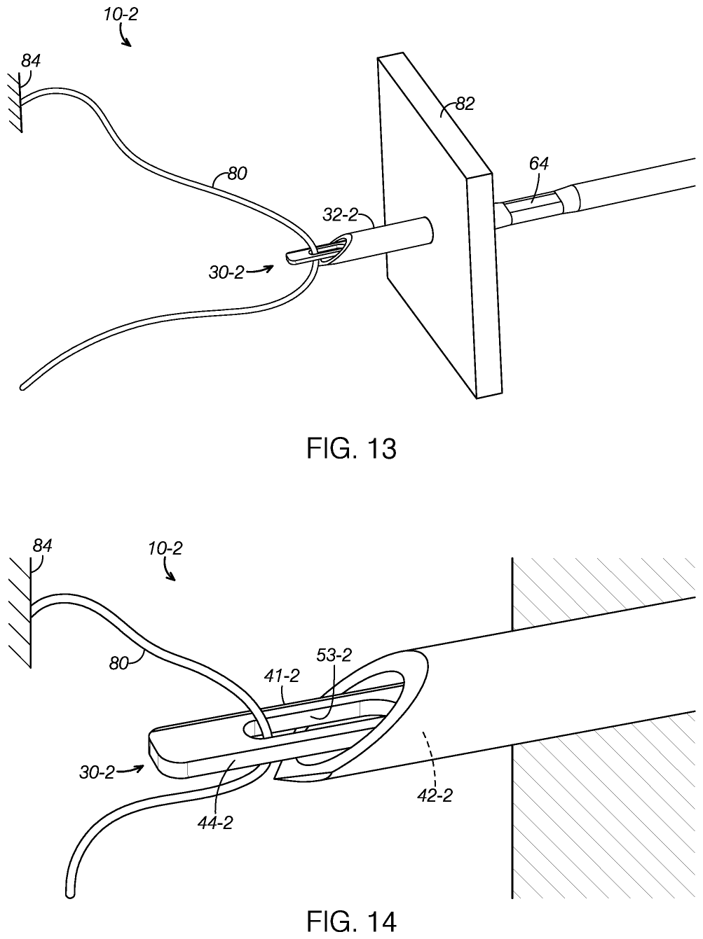

In FIG. 13, the jaw assembly 30-2 is retracted with respect to the shaft 32-2 and partially pulled into the lumen of the shaft 32-2 so as to close the jaw assembly 30-2. As shown in more detail in FIG. 14, the jaw members 41-2, 42-2 are moved by the inner walls of the shaft 32-2 to form the closed-end channel 53-2 with the suture 80 loosely captured therein. In FIGS. 13-15, the captured suture 80 can still slide loosely within the closed-end channel 53-2 even as the suture 80 is retained and carried by the jaw assembly 30-2 through the layer of tissue 82. The sliding action of the carried suture 80 within the jaw assembly 30-2 is accomplished by not having the jaw assembly 30-2 grasp onto the suture 80. In other words, the jaw assembly 30-2 can be employed in only the sliding mode, as opposed to the grasping mode, when one end of the suture is affixed or anchored to a certain object such as a bone anchor.

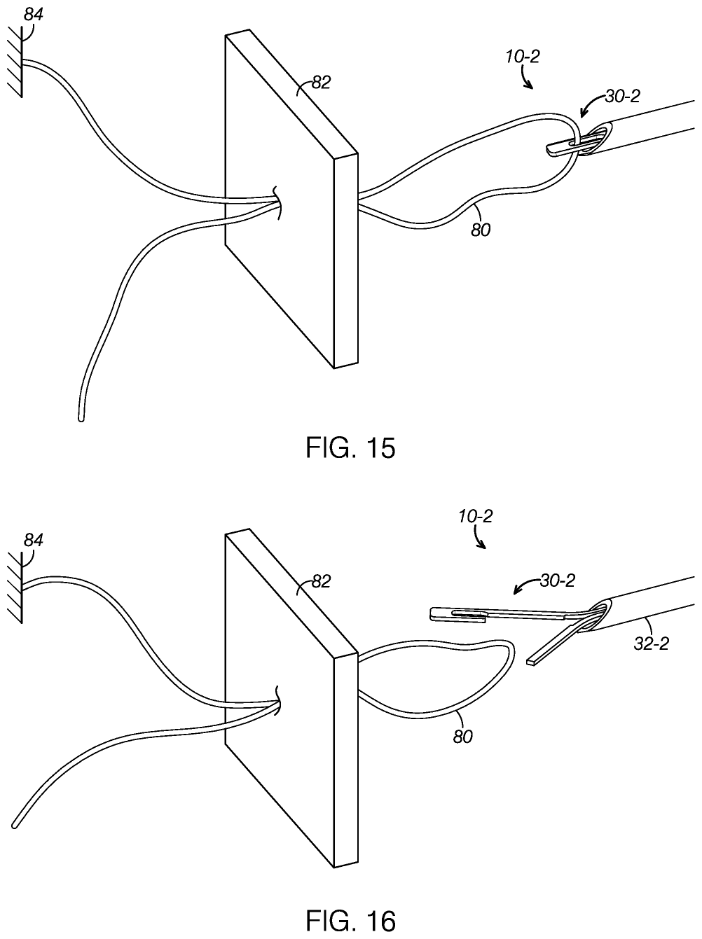

In FIG. 15, the suture passing device 10-2 is pulled back out of the tissue 82 with the loosely retained suture 80 slidingly captured within the jaw assembly 30-2. In FIG. 16, the suture 80 which has been passed through the tissue 82 may then be released by advancing the jaw assembly 30-2 distally with respect to the shaft

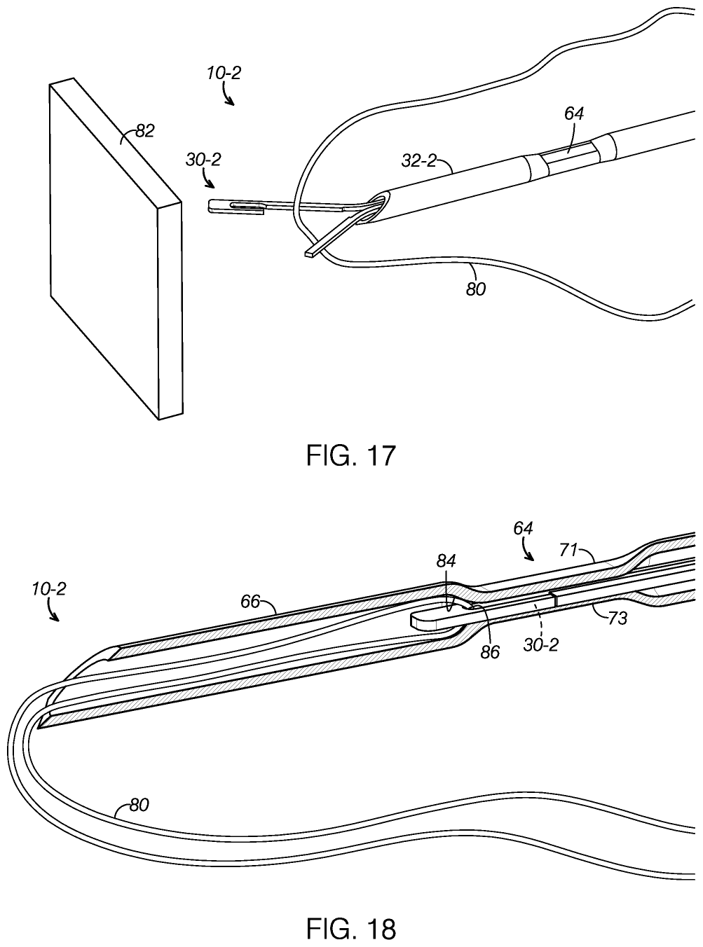

FIGS. 17 and 18 illustrate a grasping mode of the suture passing device 10-2. In FIG. 17, the jaw assembly 30-2 of the device 10-2 is moved to an open position. The suture 80 may be initially secured by the hook 44-2. The jaw assembly 30-2 may then be retracted with respect to the shaft 32-2 to close the jaw members 41-2, 42-2 as previously shown in FIGS. 13-14.

In FIG. 18, the jaw assembly 30-2 may be further retracted into the shaft 32-2 so as to pull the suture 80 into the distal shaft portion 66 to the point where the suture 80 is pulled up against the tapered wall 84 and flat sections 71, 73. Since the suture 80 is captured within the dead-end channel 53-2, the suture 80 gets pinned between the distal edge 86 of dead-end channel 43-2 and the tapered wall 84. This allows manipulation of the suture 80 with the device 10-2 since they both 80, 10-2 are temporarily fixed to each other.

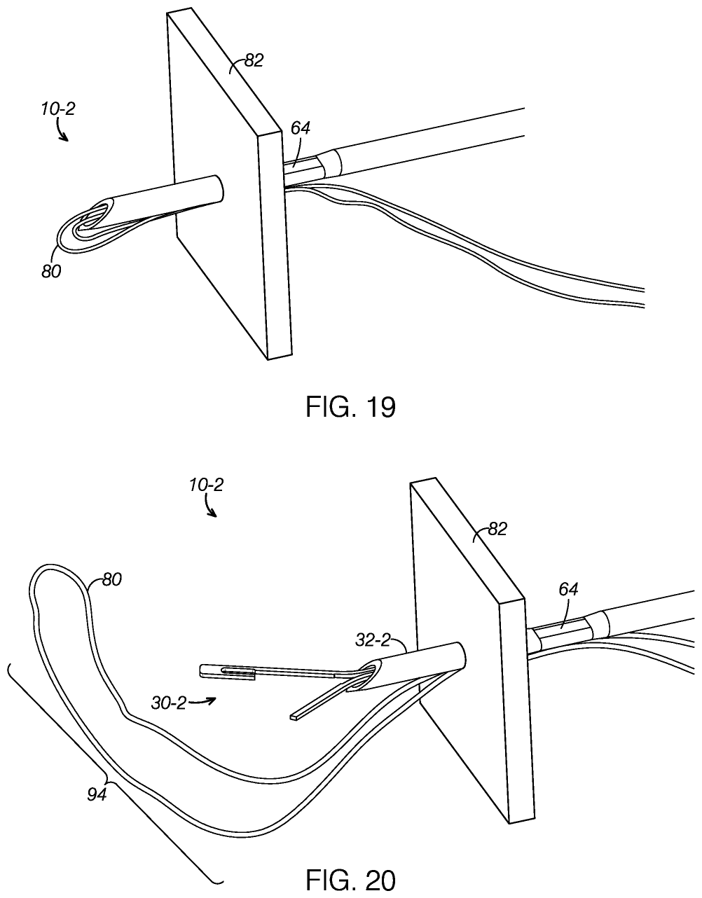

In FIG. 19, the suture passing device 10-2 with the captured 80 fixed securely within can then be pushed through the tissue 82 carrying the suture 80 therethrough. Thereafter the jaw assembly 30-2 may be deployed to spread apart the jaw members 41-2, 42-2 so that the suture 80 can drop freely from the suture passing device 10-2.

In FIG. 20, it will be appreciated that the suture passing device 10-2 deploys and leaves a significantly large bolus 94 of suture 80 after penetrating the layer of tissue 82. The large bolus is accomplished in part by the jaw assembly 30-2 pulling suture 80 more deeply within the shaft 32-2 as shown in FIG. 18. This extensive retraction of the jaw assembly 32-2 within the shaft 32-2 enables a greater bolus to be created when releasing the suture 80. After penetrating the tissue layer 82, the deeply retracted jaw assembly 30-2 with the captured suture is then pushed out to form a larger bolus 94.

It is important that faces 61 and 62 are close enough to each other during this deployment phase to be able to push the suture bolus out the tube

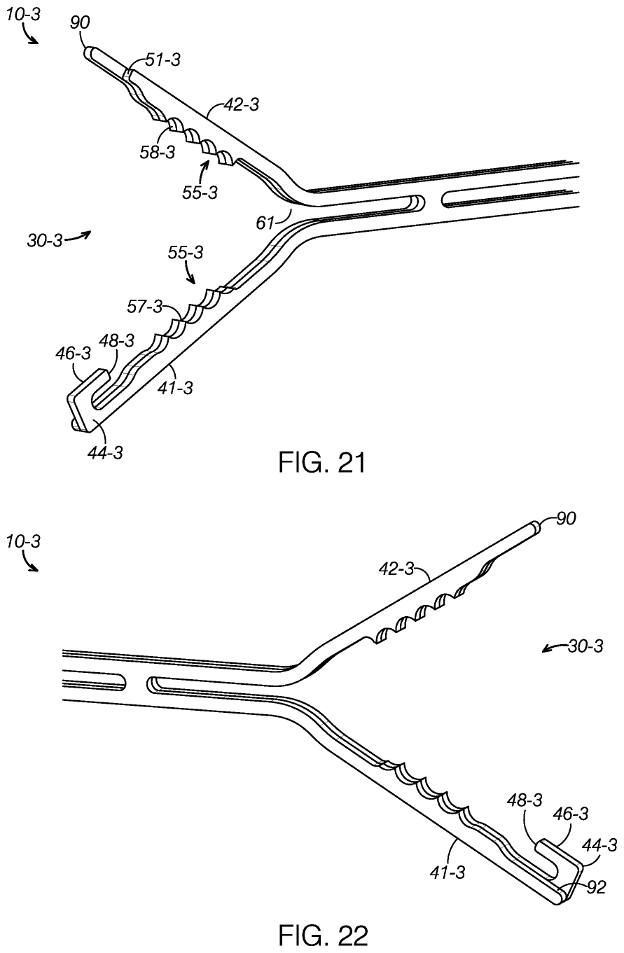

In FIG. 21, a third preferred embodiment of a suture passing device 10-3 may comprise a jaw assembly 30-3 with an extra wide width. In the illustrated embodiment, the preferred jaw assembly 30-3 comprises a double-wide first jaw member 41-3 and a double-wide second jaw member 42-3, each having a width in the range of 0.2 mm to 1 mm.

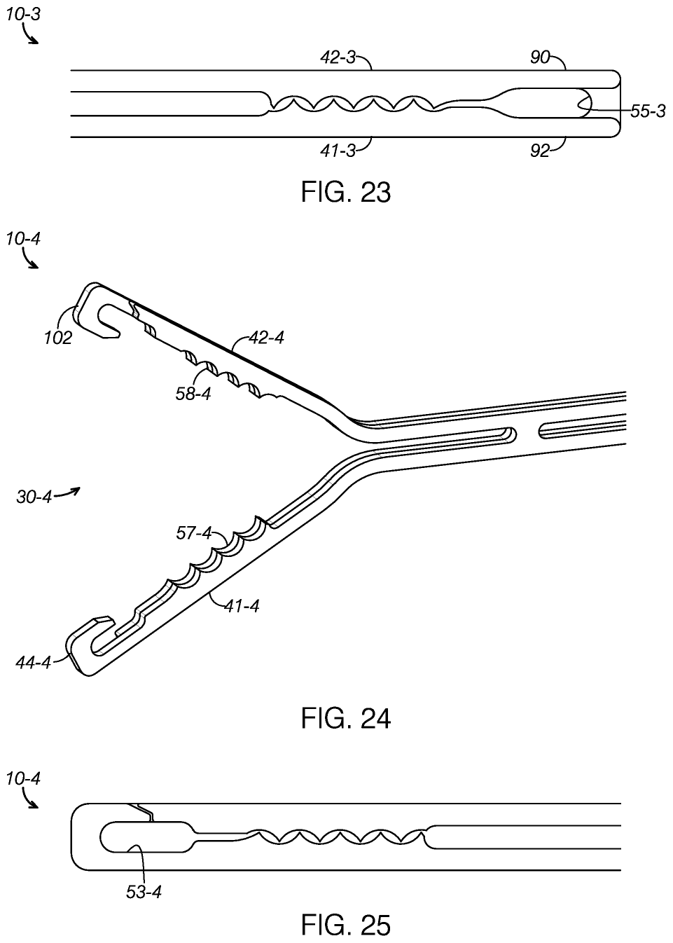

The first jaw member 41-3 preferably comprises a distal hook 44-3 with a lesser width than the width of the jaw member 41-3. In the preferred embodiment, the distal hook 44-3 comprises a return 46-3 that extends in a proximal direction and terminates at an abutting end 48-3. When the jaw assembly 30-3 is in the closed position as shown in FIG. 3, the abutting end 48-3 of the first jaw member 41 contacts an abutting end 51-3 of the second jaw member 42-3 to form a closed end channel 53 as shown more apparently in FIG. 23.

The second jaw member 42-3 comprises an extension leg 90 that protrudes distally beyond the abutting end 51-3.

In this illustrated embodiment, the suture grasping mechanism 55-3 comprises a first scalloped edge 57-3 formed on a medial side of the first jaw member 41-3 and a second scalloped edge 58-3 formed on a medial side of the second jaw member 24. The pair of scalloped edges 57, 58 are aligned with each other so as to fix on a point of secured suture when the jaw assembly 30 is in the closed position as shown in FIG. 3.

In FIG. 22, the first jaw member 41-3 comprises a distal leg 92 located laterally adjacent to the hook 44-3. In the closed position as shown in FIG. 23, the extension leg 90 of the second jaw member 42-3 and the distal leg 92 of the first jaw member 41-3 are disposed in a substantially parallel manner while the hook 44-3 forms a closed-end channel 53-3.

In FIGS. 24 and 25, a fourth preferred embodiment of a suture passing device 10-4 comprises a jaw assembly 30-4 with dual hooks 44-4, 102. In particular, a first jaw member 41-4 comprises a first hook 44-4 while a second jaw member 42-4 comprises a second hook 102.

The first jaw member 41-4 also comprises a first scalloped edge 57-4 proximal to the first hook 44-4, while the second jaw member 42-4 comprises a second scalloped edge 58-4 proximal to the second hook 102. By forming each jaw member 41-4, 42-4 with a corresponding hook 44-4, 102, respectively, a surgeon may use the suture passing device 10-4 to initially engage suture with either jaw member 41-4, 42-4. This provides the operator with greater flexibility to engage suture depending upon whichever jaw member is closer to the suture.

In the closed position as shown in FIG. 25, a closed-end channel 53-4 is formed.

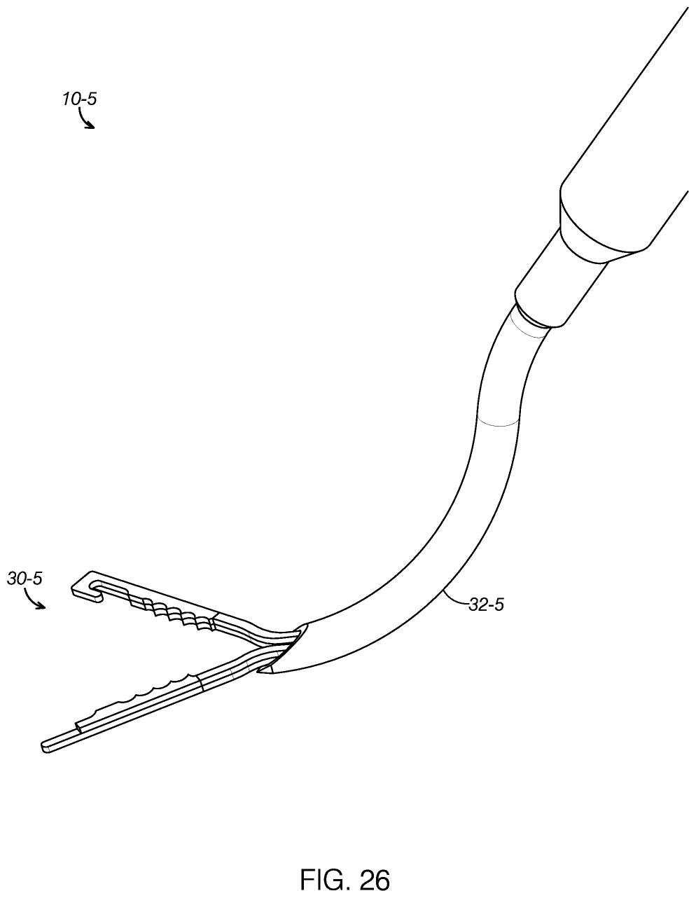

FIG. 26 illustrates an alternative embodiment of a suture passing device 10-5 having a curved shaft 32-5. The thin ribbon that comprises the jaw assembly 30-5 is flexible enough to traverse the non-linear path defined by the curved shaft 32-5.

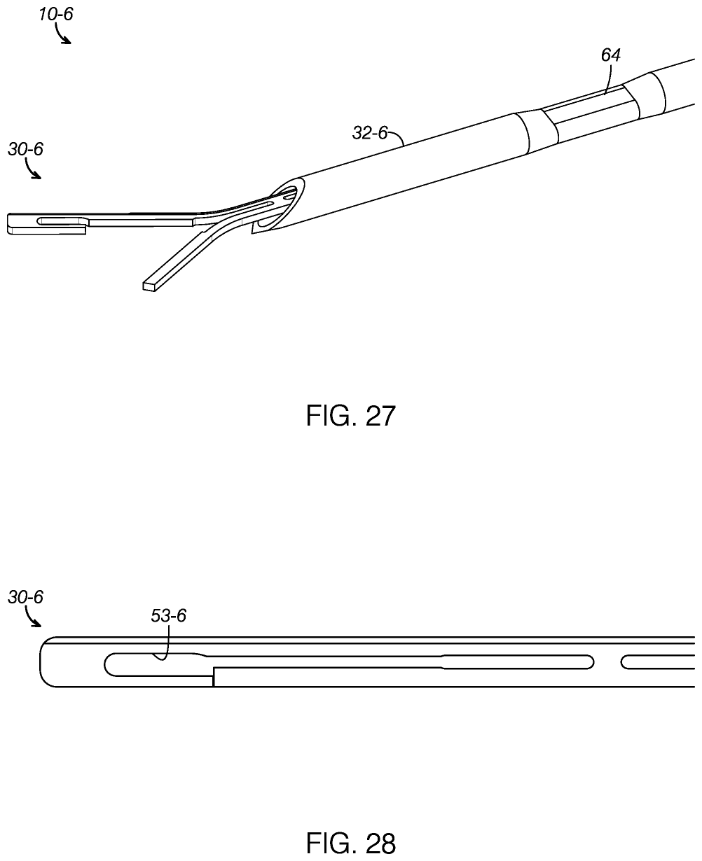

In FIG. 27, an alternative embodiment of a suture passing device 10-6 comprises a jaw assembly 30-6 that includes a closed-channel 53-6, but not a grasping mechanism. FIG. 28 illustrates the jaw assembly 30-6 in a closed position which would occur when disposed within the shaft 32-6. It will be appreciated that the jaw assembly itself omits a grasping mechanism. However, while the jaw assembly 30-6 omits a grasping mechanism, the suture passing device 10-6 may nonetheless grasp onto suture by retracting the jaw assembly 30-6 with the captured suture deep enough to cause the captured suture to be pinched against the crimped portion 64 of the shaft 32-6.

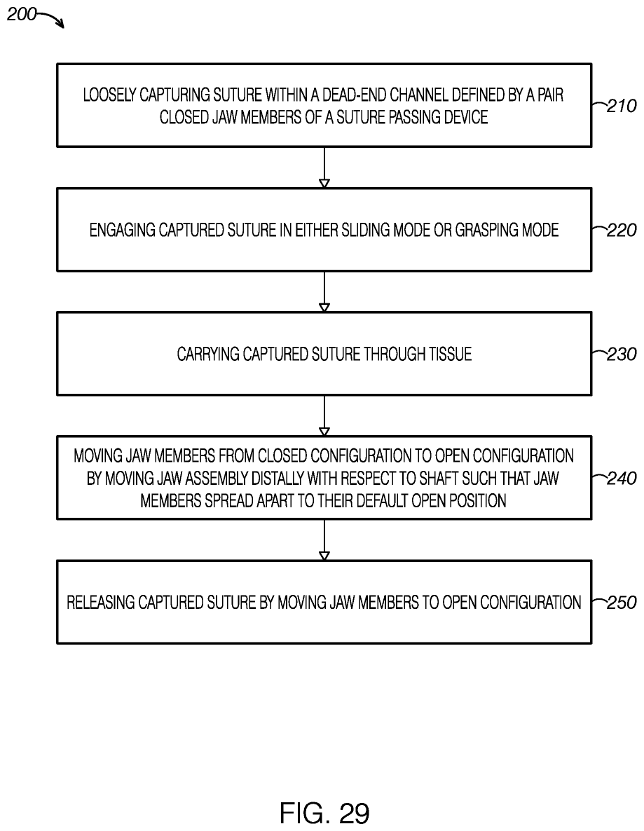

A preferred method of passing suture 200 is also provided and illustrated in FIG. 29. The method 200 comprises:
the step 210 of loosely capturing suture within a dead-end channel defined by a pair of closed jaw members of a suture passing device.
the step 220 of engaging captured suture in either sliding mode or grasping mode;
the step 230 of carrying the captured suture through tissue.

In step 240, the jaw assembly is moved distally with respect to the shaft to open the jaw members. In particular, the jaw members spread apart to their default configuration.

In step 250, the captured suture is released by the jaw assembly as the jaw members exit the shaft of the suture passing device to move into a default open configuration.

Terms such as “top,” “bottom,” “front,” “rear,” “above,” “below” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference. Similarly, an item disposed above another item may be located above or below the other item along a vertical, horizontal or diagonal direction; and an item disposed below another item may be located below or above the other item along a vertical, horizontal or diagonal direction. While some features are shown facing away from gravity, it will be understood that features can be rotated or positioned perpendicular to gravity and work to hold, knot, or cut a suture in the same way as shown.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a sub combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

1. A suture passing device, comprising:
a handle;
a shaft coupled to the handle, the shaft comprising a sharp distal tip that defines a shaft axis, the shaft defining a lumen;
a jaw assembly housed within the shaft, the jaw assembly comprising a pair of jaw members including a first jaw member and a second jaw member, the first jaw member and the second jaw member being movable with respect to each other between a closed position and an open position;
an actuating mechanism coupled to the jaw assembly and configured to move the jaw assembly between the closed position and the open position; and
a suture grasping mechanism, wherein the first jaw member and the second jaw member are biased away from each other towards the open position and moved to the open position when the actuating mechanism moves the jaw assembly distally with respect to the shaft such that the first jaw member and the second jaw member each diverge from the shaft axis when exiting the shaft, and
wherein the first jaw member and the second jaw member are moved to the closed position when the actuating mechanism is moved to retract the jaw assembly proximally with respect to the shaft,
wherein the first jaw member comprises a hook that defines a channel for receiving a portion of a suture, the channel being sized to loosely carry the suture, and
wherein the second jaw member comprises an extension leg that extends adjacent, in a width-wise direction, the hook, the width-wise direction being orthogonal a direction of movement of the second jaw member during movement between the open position and the closed position.

2. The device of claim 1, wherein:
the first jaw member comprises a first width in the range of 0.2 mm to 1 mm; and
the second jaw member comprises a second width in the range of 0.2 mm to 1 mm.

3. The device of claim 2, wherein the extension leg has an extension leg width smaller than the second width.

4. The device of claim 2, wherein the second jaw member comprises a second hook.

5. The device of claim 1, wherein the first jaw member comprises a first proximal portion having a first scalloped edge.

6. The device of claim 5, wherein the second jaw member comprises a second proximal portion having a second scalloped edge.

7. The device of claim 1, wherein the suture grasping mechanism comprises:
a first proximal jaw portion having a first flat surface;
a second proximal jaw portion having a second flat surface that contacts the first flat surface when the jaw assembly is in the closed portion, and
a crimped shaft portion proximal to the sharp distal tip.

8. The device of claim 1, wherein the hook forms a closed loop with a distal tip of the second jaw member when the jaw assembly is in the closed position.

9. A suture passing device, comprising:
a handle;
a shaft coupled to the handle, the shaft comprising a sharp distal tip that defines a shaft axis, the shaft defining a lumen;
a jaw assembly housed within the shaft, the jaw assembly comprising a pair of jaw members including a first jaw member and a second jaw member, the first jaw member and the second jaw member being movable with respect to each other between a closed position and an open position;
an actuating mechanism coupled to the jaw assembly and configured to move the jaw assembly between the closed position and the open position; and
a suture grasping mechanism,
wherein the first jaw member and the second jaw member are biased away from each other towards the open position and moved to the open position when the actuating mechanism moves the jaw assembly distally with respect to the shaft such that the first jaw member and the second jaw member each diverge from the shaft axis when exiting the shaft,
wherein the first jaw member and the second jaw member are moved to the closed position when the actuating mechanism is moved to retract the jaw assembly proximally with respect to the shaft,
wherein the first jaw member comprises a hook that forms a closed loop with a distal tip of the second jaw member when the jaw assembly is in the closed position, and wherein the second jaw member comprises an extension leg that extends adjacent, in a width-wise direction, the hook, the width-wise direction being orthogonal a direction of movement of the second jaw member during movement between the open position and the closed position.

10. The device of claim 9, wherein:
the first jaw member comprises a first width in the range of 0.2 mm to 1 mm; and
the second jaw member comprises a second width in the range of 0.2 mm to 1 mm.

11. The device of claim 10, wherein
the hook comprises a first hook; and
the extension leg creates a second hook that is laterally offset with respect to the first hook.

12. The device of claim 9, wherein the first jaw member comprises a first proximal portion having a first scalloped edge.

13. The device of claim 12, wherein the second jaw member comprises a second proximal portion having a second scalloped edge.

14. The device of claim 9, wherein the suture capturing mechanism comprises:
a first proximal jaw portion having a first flat surface;
a second proximal jaw portion having a second flat surface that contacts the first flat surface when the jaw assembly is in the closed portion; and
a crimped shaft portion proximal to the sharp distal tip.

15. A suture passing device, comprising:
a handle;
a shaft coupled to the handle, the shaft comprising a sharp distal tip that defines a shaft axis, the shaft defining a lumen;
a jaw assembly housed within the shaft, the jaw assembly comprising a pair of jaw members including a first jaw member and a second jaw member, the first jaw member and the second jaw member being movable with respect to each other between a closed position and an open position;
an actuating mechanism coupled to the jaw assembly and configured to move the jaw assembly between the closed position and the open position; and
a suture grasping mechanism,
wherein the first jaw member and the second jaw member are biased away from each other towards the open position and moved to the open position when the actuating mechanism moves the jaw assembly distally with respect to the shaft such that the first jaw member and the second jaw member each diverge from the shaft axis when exiting the shaft,
wherein the first jaw member and the second jaw member are moved to the closed position when the actuating mechanism is moved to retract the jaw assembly proximally with respect to the shaft,
wherein the first jaw member comprises a hook that forms a closed loop with a distal tip of the second jaw member when the jaw assembly is in the closed position,
wherein the first jaw member comprises a first width;
wherein the hook has a hook width that is less than the first width, the first width and the hook width being taken in a width-wise direction orthogonal a direction of movement of the first jaw member during movement between the open position and the closed position; and
wherein the second jaw member comprises a second width.

16. The device of claim 15, wherein
the hook comprises a first hook; and
the second jaw member comprises a second hook that is laterally offset with respect to the first hook.

17. The device of claim 15, wherein the first jaw member comprises a first proximal portion having a first scalloped edge.

18. The device of claim 17, wherein the second jaw member comprises a second proximal portion having a second scalloped edge.

19. The device of claim 15, wherein the suture grasping mechanism comprises:
a first proximal jaw portion having a first flat surface;
a second proximal jaw portion having a second flat surface that contacts the first flat surface when the jaw assembly is in the closed portion; and
a crimped shaft portion proximal to the sharp distal tip.

20. The device of claim 15, wherein:
the second jaw member comprises an extension leg that extends adjacent, in the width-wise direction, the hook; and
the extension leg having an extension leg width that is less than the second width.
